(19) United States
(12) Patent Application Publication
(10) Pub. No.: US 2006/0134667 A1
(43) Pub. Date: Jun. 22, 2006

(54) Method for detecting fusion gene

(76) Inventors: Masatoshi Narahara, Sayama (JP); Toshiro Saito, Hatoyama (JP); Takayuki Obara, Kodaira (JP); Kenji Yasuda, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/274,314

(22) Filed: Nov. 16, 2005

(30) Foreign Application Priority Data
Nov. 16, 2004 (JP) JP 2004-331808

(51) Int. Cl. C12Q 1/68; C12P 19/34

(52) U.S. Cl. 435/6; 435/91.2

(57) ABSTRACT

The present invention relates to a method for detecting fusion gene transcripts resulting from chromosomal translocation. Specifically, the method of the present invention comprises allowing at least two or more probes, each of which contains a partial base sequence of exons which sandwich the breakpoint of a fusion gene or complementary base sequence thereof, and each of which immobilized on a support, to hybridize with a sample containing a nucleic acid derived from a fusion gene, thereby allowing the detection of two or more fusion genes at a time.

### CLAIM OF PRIORITY

The present application claims priority from Japanese application JP 2004-331808 filed on Nov. 16, 2004, the content of which is hereby incorporated by reference into this application.

### BACKGROUND OF THE INVENTION

1. Field of the Invention

The present invention relates to a method for detecting fusion gene transcripts resulting from chromosomal translocation, more specifically a method for detecting two or more fusion genes with high throughput at a time.

2. Background Art

It is widely known that there are observed chromosomal translocations characteristic to disease types of leukemia, and it is known that fusion genes resulted from the chromosomal translocations also play an important role in the development of leukemia (Semin Hematol. 1999 October; 36(4): 401-410 A, Semin Hematol. 1999 October; 36(4): 390-400). In addition, the translocation of a chromosome is closely concerned with the classification of leukemia, selection of a therapeutic method, prognostic progress, etc., besides the development of leukemia (Leukemia. 1994 March; 8(3): 454-457, Leukemia. 1999 July; 13(7): 999-1008), and detection of translocation of chromosomes has become an indispensable item to the diagnosis of leukemia.

As a method of detecting such a translocation of chromosome on a chromosome level, FISH (Fluorescence In Situ Hybridization) method is widely known (Genes Chromosomes Cancer. 1998 June; 22(2): 87-94, Cancer Genet Cytogenet. 1998 Jul. 1; 104(1): 57-60). In FISH method, after preparing fluorescent probes having base sequences specific to each of the related genes which constitute a fusion gene on both sides of the breakpoint and labeled with different fluorescent dyes, these probes are hybridized with sample chromosome. Then, positions on the chromosome on which the different fluorescent probes are hybridized are observed using a fluorescence microscope.

On the other hand, RT-PCR (Reverse Transcription-Polymerase Chain Reaction) method using reverse transcription reaction is widely known as a method of detecting a fusion gene which is a transfer product (Leukemia. 1995 April; 9(4): 588-593). In RT-PCR method, the presence of amplification or amount of amplification is detected using a forward primer and a reverse primer designed to hybridize on both sides of the breakpoint of the fusion gene. As for the detection method, there is a method of observing the amplification product by gel electrophoresis (Leukemia. 1999 December; 13 (12): 1901-1928) and a method of using fluorescently-labeled probes (Leuk Lymphoma. 2002 December; 43 (12): 2291-2299).

Examples of the detection method of a fusion gene transcript using a forward primer and a reverse primer like RT-PCR method also include NASBA method, etc. (JP Patent Publication (Kokai) No. 10-229899, JP Patent Publication (Kokai) No. 2000-300261 A (1998)).

As a translocation of chromosome associated with leukemia, combination of no less than 29 genes has been reported. Furthermore, even if the translocating genes are of the same combination, there exist many types in which the translocation positions on the gene are different and existence of 80 or more fusion genes has been reported (Blood, July 1998; 92: 574-588). However, since the kind of fluorescent dyes which can be used in one assay in the FISH method are limited, many fusion genes associated with leukemia cannot be detected with high throughput. In addition, although the analysis of minimal residual disease (MRD) based on the quantification of fusion gene transcripts is important in the diagnosis of leukemia, the FISH method has low quantitativity and difficult for the analysis of MRD. Multiplex RT-PCR method is also proposed as a method having a higher throughput than FISH method (Blood, July 1998; 92: 574-588), this method requires detection by eight times of amplification reaction as well as electric electrophoresis, and is hard to be called as a high throughput detection method.

In the meantime, a method using Real-time PCR method was reported as a method having high quantitativity in which improvement in throughput was considered (JP Patent Publication (Kokai) No. 2002-136300 A). However, it is known that the efficiency of amplification differs with the size of the amplification product in the Real-time PCR method, and in order to increase the quantitativity, it is necessary to adjust the size of amplification product to some extent. Here, since the kind of the target fusion gene is identified based on the size of the amplification product to be amplified in the Real-time PCR method, the forward primer and reverse primer should be designed so that the size of the amplification product differs for every fusion gene while the size of the amplification product of the gene to be detected is adjusted to some extent. Therefore, the more the number of genes to be detected increases, the less the flexibility of primer design becomes, and as a result it becomes impossible to design a primer set which detects simultaneously as many as 80 fusion genes. Furthermore, fluorescent probes required for detection are also required along with enzymes required for amplification in the Real-time PCR method, and therefore there also arises a problem that the test cost becomes high. Also in the NASBA method, similar problems as in the Real-time PCR method in throughput and quantitativity are present.

An object of the present invention is to provide a method for detecting two or more fusion genes with high throughput at a time without using PCR method.

### SUMMARY OF THE INVENTION

In order to attain the object, the present invention allows at least two or more probes, each of which contains a partial base sequence of exons which sandwich the breakpoint of a fusion gene or complementary base sequence thereof, and each of which is immobilized on a support, to hybridize with a sample which contains a nucleic acid (amplification product) derived from the fusion gene. Since a number of probes corresponding to the partial base sequence in each of the exon sequence are immobilized on the support, two or more fusion genes can be detected at a time.

Moreover, the method for detecting fusion gene transcripts of the present invention enables to detect two or more fusion genes at a time which are identical in the combination of translocated genes but different from each other in the translocated position in the genes by labeling the nucleic acids in the sample beforehand and analyzing the signal intensity from the nucleic acid hybridized with each probe.

The sample containing the nucleic acids derived from the fusion gene can be prepared, for example, by the following steps (1) to (4) in the method for detecting fusion gene transcripts of the present invention:

(1) a step of synthesizing a single stranded DNA by subjecting RNA obtained from a specimen from subject to reverse transcription reaction;

(2) a step of synthesizing a double stranded DNA by using the single stranded DNA as a template;

(3) a step of amplifying cRNA using RNA polymerase by using the double stranded DNA as a template; and

(4) a step of synthesizing a single stranded DNA by performing reverse transcription reaction by using the cRNA as a template.

In the reverse transcription reaction of the step (1), for a base sequence containing at least ten or more contiguous bases which exist on the 3′ side from the breakpoint of the fusion gene, a primer containing a base sequence complementary to this primer is used. In order to improve the amplification efficiency of cRNA, it is preferable to bind a promoter sequence of RNA polymerase to this primer.

In addition, a primer containing at least ten or more contiguous bases which exist on the 5′ side from the breakpoint of the fusion gene is used in the reverse transcription reaction of the step (4).

Since the present invention uses such sequence specific primers and only amplifies the product derived from the genes required for detection, noise during the detection can be reduced.

The present invention also provides a kit used for the method for detecting fusion gene transcripts in the present invention. Although the kit of the present invention comprises the following (a) to (c) as essential constituent elements, it may contain other reagents required for detection, such as an enzyme, a substrate and a reagent for detection, etc. if needed.

(a) a support on which two or more probes containing a partial base sequence of exons which sandwich the breakpoint of a fusion gene or complementary base sequence thereof are immobilized;

(b) a first primer containing a base sequence complementary to a base sequence of at least ten or more contiguous bases which exist on the 3′ side from the breakpoint of the fusion gene; and

(c) a second primer containing a base sequence of at least ten or more contiguous bases which exist on the 5′ side from the breakpoint of the fusion gene.

The method and the kit for detecting fusion gene transcripts of the present invention can be used for diagnosis of diseases closely associated with generation of a fusion gene, for example, leukemia (the classification of leukemia, selection of a therapeutic method, prognostic progress, etc.). In the detection of the fusion gene transcript associated with leukemia, the first primer containing a probe which contains the base sequences as shown in SEQ ID NOs: 1 to 51 and the base sequence as shown in SEQ ID NOs: 52 to 64, and the second primer as shown in SEQ ID NOs: 65 to 77 can be used as the probe or primer.

According to the method for detecting fusion gene transcripts of the present invention, two or more fusion genes can be detected at a time with a high throughput.

### BRIEF DESCRIPTION OF THE DRAWINGS

FIG. 1 is a schematic view showing the structure of each exon which constitutes BCR, ABL and a BCR/ABL fusion gene; and

FIG. 2 a schematic view showing the support used for the detection of BCR/ABL fusion gene and probes immobilized on the support in the present invention.

### DETAILED DESCRIPTION OF THE PREFERRED EMBODIMENTS

The present invention will be described in detail hereafter.

In the present invention, after allowing at least two or more probes, each of which contains a partial base sequence in exons which sandwich the breakpoint of a fusion gene or complementary base sequences thereof, and each of which is immobilized on a support, to hybridize with a sample which contains a nucleic acid amplification product derived from at least two or more fusion genes, the kind of fusion genes which exist in the sample is identified using the obtained signal intensity. In the present invention, not only the combination of the gene which constitutes the fusion gene but the kind of various fusion genes which are the same in combination and different in translocation position can be simultaneously detected.

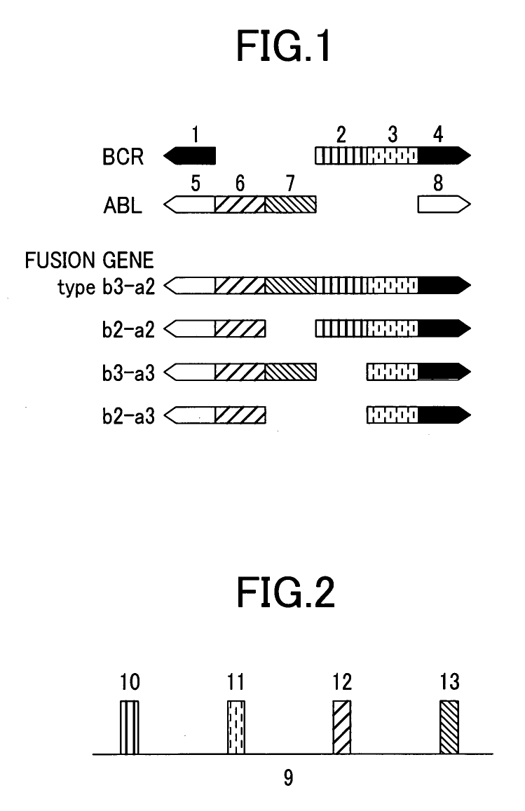

Details are explained below using an example of the fusion gene of BCR and ABL resulting from chromosomal translocation in t (9; 22) (q34; q11). As shown in FIG. 1, four fusion genes exist in the chromosomal translocation between the BCR and the ABL. Therefore, it is usually necessary in an actual clinical sample to identify the type of the fusion genes which exists in the sample in a system in which the BCR and the ABL which are not fusion genes but normal types and one kind of fusion gene of an unknown type coexist.

In the present invention, as shown in FIG. 2, partial base sequences in the exon sequence of the fusion gene or base sequences complementary thereto are immobilized in different specific positions on the support, and hybridized to the product derived from the clinical sample. And the type of the fusion genes which exist in the sample are identified using the signal intensity obtained from the areas where each of the probes is immobilized.

Here, for the case other than the translocation of the t (9; 22) (q34; q11), if the similar partial base sequences in the exon or base sequences complementary thereto are immobilized in different positions on the support, two or more fusion genes can be detected at a time.

Furthermore, in the present invention, fusion gene transcripts can be detected using the nucleic acid amplification products prepared from various specimens from subjects by the following steps (1) to (4):

(1) a step of synthesizing a single stranded DNA by subjecting RNA obtained from a specimen from subject to reverse transcription reaction;

(2) a step of synthesizing a double stranded DNA by using the single stranded DNA as a template;

(3) a step of amplifying cRNA using RNA polymerase by using the double stranded DNA as a template; and

(4) a step of synthesizing single stranded DNA by performing reverse transcription reaction by using the cRNA as a template.

A single stranded DNA is synthesized using primers having base sequences complementary to the fusion gene and a reverse transcriptase in step (1). It is preferable to have a promoter sequence in the sequence of primer used at this time for allowing the RNA polymerase to act.

As such a promoter sequence, 5′-AATTGTAATACGACTCACTATAGGG-3′ (SEQ ID NO: 78) can be used if the polymerase to be used is T7RNA polymerase. The promoter sequence used may contain a spacer sequence to the replication origin which follows. For example, 5′-AGGAGAG-3′ is known as a spacer sequence and a part thereof may be linked to the 3′ end of the promoter sequence if necessary. Amplification efficiency can be improved by inserting a spacer sequence depending on the region to be amplified. Examples of the other promoter sequence include 5′-ATTAACCCTCACTAAAG-3′ (SEQ ID NO: 79) for T3RNA polymerase and 5′-ATTTAGGTGACACTATA-3′ (SEQ ID NO: 80) for SP6RNA polymerase. Particularly, it is preferable to use T7RNA polymerase and its promoter sequence in the present invention.

A polyT sequence which can be used commonly for all the fusion genes can also be used, but use of a primer which includes a specific sequence designed for each of the fusion genes is particularly preferable. By using primer having such a specific sequence, only a product derived from the gene to be analyzed can be amplified specifically, and the noise during detection can be reduced.

In step the (2), DNA polymerase is acted on the single stranded DNA obtained in the step (1) as a template, and a double stranded DNA is synthesized. Since the promoter sequence of RNA polymerase exists at the end of the obtained double stranded DNA, transfer reaction proceeds and cRNA can be amplified through the action of RNA polymerase in the step (3).

Finally, in the step (4), reverse transcription reaction is performed using cRNA obtained in the step (3) as a template, and a single stranded DNA is synthesized. Although a random hexamer sequence which can be used commonly for all the fusion genes and specific sequences designed for each of the fusion genes as a primer sequence used, it is preferable to use a primer including a specific sequence designed for each of the fusion genes.

Both the step (1) and step (4) can suppress generation of normal genes which are not fusion genes such as BCR or ABL as shown in FIG. 1 by using a primer which has a specific sequence, and can drastically reduce the noise during detection.

In addition, according to the method of the present invention, WT1 (Blood, November 1994; 84:3071-3079) which is a leukemia related gene other than fusion gene and GAPD (GlycerAldehyde-3-Phosphate Dehydrogenase), ACTB (Actin, beta), etc. which are endogenous control genes can be simultaneously detected. The amount of expression of the fusion gene contained in the sample can be quantified, which enables to analyze MRD, by comparing the signal intensity thus obtained from an endogenous control gene and the signal intensity derived from the fusion gene. Alternatively, analysis of MRD can be performed by measuring the signal intensity obtained after mixing an exogenous sample separately and the signal intensity derived from the fusion gene for the purpose of improvement in quantitativity.

The detection method of the nucleic acid hybridized to the probe is not particularly limited, but fluorescence, phosphorescence, luminescence, or radioisotope, etc. can be suitably used. When the fluorescence detection is used, a method in which fluorescently labeled bases are introduced into the nucleic acids at the time of the reverse transcription reaction of the step (4) or a method in which a fluorescent substance in which N-hydroxysuccinimide group etc. has been introduced is reacted with a product obtained after a suitable functional group represented by aminoallyl group is allowed to be incorporated thereby to effect labeling, etc. can be used. Alternatively, a method of labeling with an alkylating agent such as cyclophosphamide can be used for the product obtained in the step (4). Furthermore, as a detection method not using labeled product, a method in which a special compound is intercalated into the double stranded DNA after hybridization and the compound is detected by luminescence or electrically and so on can be used.

There is no particularly limitation on the sequences immobilized on the support as long as they include a partial base sequence of exons of the fusion gene or base sequence complementary thereto in the sequence. Generally, in hybridization of a base sequence immobilized on the support and the product in the solution, the closer to the support, the more significantly decreases hybridization efficiency due to the steric obstacle. Therefore, a spacer sequence like polyT sequence may be inserted in the portion near the support for the purpose of improving hybridization efficiency.

Examples of the material of the support used in the present invention include one or more members selected from plastic, inorganic high polymer, metal, natural high polymer and ceramics. As a plastic, specifically polyethylene, polystyrene, polycarbonate, polypropylene, polyamide, phenol resin, epoxy resin, polycarbodiimide resin, polyvinyl chloride, polyvinylidene fluoride, polyfluoroethylene, polyimide, acrylic resin, etc. can be exemplified and as an inorganic high polymer glass, crystal, carbon, silica gel and graphite, as a metal, solid metal at normal temperature such as gold, platinum, silver, copper, iron, aluminum and a magnet, and as ceramics, alumina, silica, silicon carbide, silicon nitride, carbonization boron, etc.

There is also no particularly limitation on the form of the support, and it is preferable to use a board-like support in order to use commercial detection equipment as it is in detecting the signal intensity after hybridization. In addition, a particulate support, a support on which detailed processing has been given on the surface can be used for the purpose of improving hybridization efficiency.

There is also no particularly limitation on the method of immobilizing partial sequences on the support. Various methods such as a method using physical adsorption (Genome Res. 1996 July; 6(7): 639-45.), a method of immobilizing by the covalent bonding using a Linking reagent (JP Patent Publication (Kokai) No. 2002-204693), or a method using a specific interaction of a thiol group and gold (J. Am. Chem. Soc. 1997; 119 (38); 8916-8920.) can be used.

### EXAMPLES

The present invention will be described more in detail by way of examples below.

### Example 1

1. Design and Synthesis of Probes

The probes having a partial base sequence of the exons of each fusion gene for the chromosome translocation to be detected using the method shown in JP Patent Publication (Kokai) No. 2003-052385 were designed (Table 1). After oligo nucleotides were synthesized using a DNA automatic synthesizer (a product of Applied Biosystem, model 394 DNA synthesizer) according to the designed probe sequences, they were purified by high-speed liquid chromatography and the probes used in the present invention were prepared.

TABLE 1

Probe Sequences used for Detection

| | | |
|---|---|---|
| Chromosomal | Gene | Sequence |

| | | | | | |
|---|---|---|---|---|---|
| Translocation | Name | Exon | Genbank | Probe Sequence | Listing |

| | | | | | |
|---|---|---|---|---|---|
| t (9; 22) | BCR/ABL | BCR_e13 | X02596 | CTTCCTTATTGATGGTCAGCGGAATGCTGTGGACAGTCTGGAGTTTCACA | SEQ ID NO:1 |
| (q34; q11) | p210 | | | CACGAGTTGGTCAGCATCTGCAGCTCCACG | |
| | | BCR_e14 | X02596 | TTGAACTCTGCTTAAATCCAGTGGCTGAGTGGACGATGACATTCAGAAAC | SEQ ID NO:2 |
| | | | | CCATAGAGCCCCGGAGACTCATCATTTTTT | |
| | | ABL_e2 | X16416 | TCCACTGGCCACAAAATCATACAGTGCAACGAAAAGGTTGGGGTCATTTT | SEQ ID NO:3 |
| | | | | CACTGGGTCCAGCGAGAAGGTTTTCCTTGG | |
| | | ABL_e3 | X16416 | ACTGGCGTGATGTAGTTGCTTGGGACCCAGCCTTGGCCATTTTTGGTTTG | SEQ ID NO:4 |
| | | | | GGCTTCACACCATTCCCCATTGTGATTATA | |
| | BCR/ABL | BCR_e1 | X02596 | TGCTGCTGTCGAAGGACTGTTGCGAGTTCTGCGAGGGAGAGCGGCTTTTG | SEQ ID NO:5 |
| | p190 | | | TCCCGGAACATGCGGTAGGTGGTGGGGCTT | |
| | | ABL_e2 | as above | as above | as above |
| | | ABL_e3 | as above | as above | as above |
| | BCR/ABL | BCR_e19 | X02596 | TGACGTCGAAGGCTGCCTTCAGTGCCTGGATGTCCGTGGCCACACCGGAC | SEQ ID NO:6 |
| | p230 | | | ACGCGGTAGATGCCCACCTCCTCCATGCCT | |
| | | BCR_e20 | X02596 | GCTCACGGAAGTACAGCTTCAGCGTGCCTGCGATGGCGTTCACGTCCATC | SEQ ID NO:7 |
| | | | | TCGCTCATCATCACCGACACATCCTTGTTA | |
| | | ABL_e2 | as above | as above | as above |
| | | ABL_e3 | as above | as above | as above |
| t (15; 17) | PML/ | PML_e3 | M73778 | CTTTCCCCTGGGTGATGCAAGAGCTGAGGTCCTGCAGGCGCACCTTGAAC | SEQ ID NO:8 |
| (q22; q21) | RARA | | | TCGTCGAAGCCATCGGTGCGCACGGCAGCT | |
| | | PML_e4 | M73778 | GCAGGTCAACGTCAATAGGGTCCCTGGGAGTGCTGGCAGCCTCTGGGCTG | SEQ ID NO:9 |
| | | | | GCTTTCTTGGATACAGCTGCATTTTTTTTT | |
| | | PML_e5 | M73778 | GCCTTTGATGGAGAAGGCGTACACTGGCACGGGGTGTGCCCCGGGCACTG | SEQ ID NO:10 |
| | | | | ACTGTACCACAGCCATAGGCTGGGCTTCAG | |
| | | PML_e6 | M73778 | TGACCTTCCTGGGGCACTGGGTCTGGCTGCACTTCCTCTTCTGGGCTGTC | SEQ ID NO:11 |
| | | | | GTTGTATTGGAGACATCTTTTTTTTTTTTT | |
| | | PML_e6_1 | M73778 | GTTGCTGTTGGGCAGGAAGACCTCACTTCCTATGACGGGGCTCCTGGGGC | SEQ ID NO:12 |
| | | | | TAGGCGGTCCATCCAGGTGGGGTGGTGAGA | |
| | | RARA_e3 | X06538 | GCTTGTAGATGCGGGGTAGAGGGGGTGGCGAGGGAGGGCTGGGCACTATC | SEQ ID NO:13 |
| | | | | TCTTCAGAACTGCTGCTCTGGGTCTCAATG | |
| t (1; 19) | E2A/ | E2A_e13 | M31222 | ACTGTAGGAGTCGGGAGGCCGAGACAGGTCAGGGAGGGTGCCTGGCTGG | SEQ ID NO:14 |
| (q23; p13) | PBX1 | | | CTGGGGAGGGCCGCGTGGTTGTGCATGAGGC | |
| | | E2A_e13_1 | M31222 | AGGTTCCAGGTGACCGAACACTTTCATTTTTTTTTTTTTTTTTTTTTTT | SEQ ID NO:15 |
| | | | | TTTTTTTTTTTTTTTTTTTTTTTTTTTTTTT | |
| | | PBX1_e2 | M86546 | ACGTGGGTGGTGAACTCGTTGCAGGCCTGCTCGTATTTCTCCAGCTCCG | SEQ ID NO:16 |
| | | | | TATGGTAGATTTGTCTGATCTGTGAGAGTTT | |
| t (4; 11) | MLL/AF4 | MLL_e8 | L04284 | CTGATTCTGGTGGTGGAGGCTGCTTTTTCTTGGGCTCACTAGGAGTGGT | SEQ ID NO:17 |
| (q21; q23) | | | | TTTGGGAACTTCTTTTCTTGGCGGTCCTGTA | |
| | | MLL_e9 | L04284 | CTTTTCTTTTGGTTTTTGTTTTACAGGGATACTTGGGCGGGGAGCCACT | SEQ ID NO:18 |
| | | | | TTTTTCTGTTTGCTCTGCTCTGGACTTTTTT | |
| | | MLL_e10 | L04284 | TGCTTAGAACTATTGCCATTGGAGAGAGTGCTGAGGATGTTCAAAGTGC | SEQ ID NO:19 |
| | | | | CTGCATTCTCCTGCTTATTGACCGGAGGTGC | |
| | | MLL_e11 | L04284 | TGCCCACTACTGGCACAGAGAAAGCAAACCACCCTGGGTGTTATAGGAA | SEQ ID NO:20 |
| | | | | CAGAAGTCAAGATTCCTAAGCCTCCCATCTC | |
| | | AF4_e4 | L13773 | CCTTCAGAATCTCTTCAACACAATGGACTTCATTGGAGTAGGTCTGTTT | SEQ ID NO:21 |
| | | | | TTTTTTTTTTTTTTTTTTTTTTTTTTTTTTT | |
| | | AF4_e5 | L13773 | TTGTAGGGAAAGGAAACTTGGATGGCTCAGCTGTACTAGGCGTATGTAT | SEQ ID NO:22 |
| | | | | TGCTGTCAAAGGAGGCGGCCATGAATGGGTC | |
| | | AF4_e6 | L13773 | TTTTGGTTTTGGGTTACAGAACTGACATGCTGAGAGTTTTTTTTTTTTT | SEQ ID NO:23 |
| | | | | TTTTTTTTTTTTTTTTTTTTTTTTTTTTTTT | |
| | | AF4_e7 | L13773 | TCACTGTCACTGTCCTCACTGTCACTGAGCTGAAGGTCGTCTTCGAGCA | SEQ ID NO:24 |
| | | | | TGGATGACGTTCCTTGCTGAGAATTTGAGTG | |
| t (9; 11) | MLL/AF9 | MLL_e8 | as above | as above | as above |
| (p22; q23) | | MLL_e9 | as above | as above | as above |
| | | MLL_e10 | as above | as above | as above |
| | | MLL_e11 | as above | as above | as above |
| | | AF9_e5 | as above | GACCTTGTTGCCTGGTCTGGGATGGTGTGAAGCTGGAGCTGGAGCTGGA | SEQ ID NO:25 |
| | | | | GCTGGAGCTGGCAGGACTGGGTTGTTCAGAT | |
| | | AF9_e6 | L13744 | ACTCTGCGACTTCGGCTGCCTCCTCTATTTACAGGCCTCTCCATTTCAG | SEQ ID NO:26 |
| | | | | AGTCATTGTCGTTATCCTCCACTTCATCTGA | |
| | | AF9_e7 | L13744 | GTTGGTTTTTAGTAAGGGTGGTGGAGGTTCGTGATGTAGGGGTGAAGAA | SEQ ID NO:27 |
| | | | | GCAGAACTGCTTTCACTATCGCTGCCATCAC | |
| | | AF9_e8 | L13744 | CCTTGTCACATTCACCATTCTTTATTTGCTTATCTGATTTGCTTTGCTT | SEQ ID NO:28 |
| | | | | TATTGGACTTTTCACTTCAAGAATTTTTTTT | |
| | | AF9_e9 | L13744 | AAGGTTCACGATCTGCTGCAGAATGTGTCTTTCTCTCAATGTCATTAAC | SEQ ID NO:29 |
| | | | | CTTCTGTGAAGCTCTACCAGTTCATCTAGGT | |
| t (8; 21) | AML1/ | AML1_e5 | D43969 | CACTGTGATTTTGATGGCTCTGTGGTAGGTGGCGACTTGCGGTGGGTTT | SEQ ID NO:30 |
| (q22; q22) | ETO | | | GTGAAGACAGTGATGGTCAGAGTGAAGCTTT | |
| | | ETO_e2 | D14289 | TTGTCGGTGTAAATGAACTGGTTCTTGGAGCTCCTTGAGTAGTTGGGGG | SEQ ID NO:31 |
| | | | | AGGTGGCATTGTTGGAGGAGTCAGCCTAGAT | |
| t (12; 21) | TEL/ | TEL_e5 | U11732 | GTGATTTGTCGTGATAGGTGACCTGGAGCGGTGCAACAGTTCAATGGTG | SEQ ID NO:32 |
| (p13; q22) | AML1 | | | GGAGGGTTATGGTGCACATTATCCACGGATG | |
| | | AML1_e2 | D43969 | TCGTGGACGTCTCTAGAAGGATTCATTCCAAGTATGCATTTTTTTTTTT | SEQ ID NO:33 |
| | | | | TTTTTTTTTTTTTTTTTTTTTTTTTTTTTTT | |
| | | AML1_e3 | D43969 | TCGGTGCGCACCAGCTCGCCCGGGTGGTCGGCCAGCACCTCCACCATGC | SEQ ID NO:34 |
| | | | | TGCGGTCGCCGCTCCTCAGCTTGCCGGCCAG | |
| inv (16) | CBFB/ | CBFB_e4 | L20298 | TGGGCTCGCTCCTCATCAAACTCCAGACAGCCCATACCATCCAGTCTTT | SEQ ID NO:35 |
| (p13; q22) | MYH11 | | | GGAGATCAATCCAGCCTTTCCAGATAACACA | |
| | | CBFB_e5 | L20298 | CTCCATTTCCTCCCGATGAGACCTGTCTCTATCTTCAAATTCGCGTGTC | SEQ ID NO:36 |
| | | | | CTTCTCCGAGCCTCTTCAAAGGCCTGTTGTG | |
| | | MYH11_e7 | D10667 | TGGCCCAGGACCCGCAGCTCCCCGGCCAGGTCTGCGTTCTCTTTCTCCA | SEQ ID NO:37 |
| | | | | GCGTCTGCTTATTCTTGTCTAGGTTCGCCTT | |
| | | MYH11_e7_1 | D10667 | TCTGCAGCTTGTGGACTTTGTCATTGAGCTCCGCCCGGGCCCGCTCCCC | SEQ ID NO:38 |
| | | | | ATCGCTGCACTTGGACTGCAGCTCCTGCACC | |
| | | MYH11_e8 | D10667 | ACTGAGGGACGCCACGTCCTTGGCCAGCTTAATGGCCTTCCCCTCGGCC | SEQ ID NO:39 |
| | | | | TCGTTAAGCATCCCTGTGACGCTCTCAACTT | |
| | | MYH11_e9 | D10667 | GTCTTGCAGGCTGTTCCGCTCCTCCTCCAGCTGGCGCAGCTTCGTAGAC | SEQ ID NO:40 |
| | | | | ACGTTGAGCTTCTGCCGGGTTTCTTCTTGAA | |
| | | MYH11_e10 | D10667 | TCTTCTTCCCCTCTTCCAGAGCTTCCACGGTGCTGGCAAAGTCCTGCAG | SEQ ID NO:41 |
| | | | | CTTCTTCTTCGAGTCGGAGATTTTTTTTTTT | |
| | | MYH11_e10_1 | D10667 | GGTTGTCCAAATCAACAACCAGGTCGTCCAGCTCCTGCTGAAGCCTGTT | SEQ ID NO:42 |
| | | | | CTTGGTCTTTTCCAGTTTATCATAAGCGGCC | |
| | | MYH11_e11 | D10667 | GTTTCCTTCTCCCTGGCTTCTGCCTCAGCTCTGTCCCTCTCATCCGCGT | SEQ ID NO:43 |
| | | | | ATTTGGAAGAGATGTTTTTCTCCTCGGCTAA | |
| | | MYH11_e12 | D10667 | CTCCTCTTCTCCTCATTCTGCTCGTCCCGGGCTTGGAGATCCCTTTCGA | SEQ ID NO:44 |
| | | | | ACTGGCCCTTGAGCGCCTGCATGTTGACTTC | |
| | | MYH11_e13 | D10667 | TTCAGGTCCCCTTCCAGCTTCTTCTTTGCTGCAGCTGCCAGGGCACGTT | SEQ ID NO:45 |
| | | | | CGTTTCGCTCGTCTTCCAGTTCCGTCTCATA | |
| 1p32 | SIL/ | SIL_e1a | M74558 | GAGCCGGCTCCAAGGAGCGCCACCGCCGACTCCGGCCCCGCCTCTGGGA | SEQ ID NO:46 |
| | TAL1 | | | CGTTGGGGTCGCGGGAACTACGTGTTTAAGT | |
| | | SIL_e1b | M74558 | CAGCATTTTGGGAGGCCGAGGCGGGTGGATCACCTGAAGTCAGGATTTC | SEQ ID NO:47 |
| | | | | CAGACCAGCCCGACCAACATGGTGAAACCCC | |
| | | TAL1_e3 | S53245 | CATATTTAGAGAGACCGGCCCCTCTGAATAGGATCTCCACTCCGCCGGA | SEQ ID NO:48 |
| | | | | AAGGGGCGGAAGCCGAGGAAGAGGATGCACA | |
| | | TAL1_e4 | S53245 | CGCGTCGCGGCCCTTTAAGTCTCTCGCGGCGCCGCCCCCACCGGCAGGG | SEQ ID NO:49 |
| | | | | CCGCCCCCCGGGCCTCCGCGCGCGCCCAGTT | |
| — | GAPD | GAPD | X01677 | ATACTTTATTAGATGGATACATGACAAGGTGCGGCTCCCATAGGCCCCT | SEQ ID NO:50 |
| | | | | CCCCTCTTCAAGGGGTCTACATGGCAACTGT | |
| — | ACTB | ACTB | X00351 | TGCATTACATAATTTACACGAAAGCAATGCTATCACCTCCCCTGTGTGG | SEQ ID NO:51 |
| | | | | ACTTGGGAGAGGACTGGGCCATTCTCCTTAG | |

2. Immobilization of Probes

A commercially available slide (a product of Gold Seal Brand) was soaked in an alkali solution (sodium hydroxide; 50 g, distilled water; 150 ml, 95% ethanol; 200 ml) at room temperature for 2 hours. Next, the slide was transferred into distilled water, rinsed 3 times, and the alkali solution was removed completely. Then, after soaking the washed slide in 10% of poly-L-lysine (a product of SIGMA) solution for one hour, the slide was drew out, centrifuged at 500 r.p.m. for one minute using a centrifuge for microtiter plate, and the poly-L-lysine solution was removed. Then, the slide was put in a suction type thermostatic chamber, dried for five minutes at 40° C., and the slide on which poly-L-lysine was introduced was prepared. The probes were immobilized at predetermined positions on the obtained slide using spotting equipment (SPBIO 2000; manufactured by Hitachi Software Engineering Co., Ltd.). At the last, after the slide was subjected to 60 mJ irradiation by a UV crosslink machine, it was dipped in a blocking treatment liquid (succinic anhydride; 5 g, N-methyl-pyrrolizinone; 315 ml, 0.2 M sodium tetraborate; 35 ml) for 15 minutes and then dipped in 95% ethanol for one minute, centrifuged at 500 r.p.m. for one minute using a centrifuge for microtiter plate, and the ethanol on the slide was removed.

3. Preparation of Labeled Products

(1) Preparation of RNA

Total RNA was extracted from K562 strain (a cell line derived from chronic myelocytic leukemia) using TRIzol reagent (a product of Invitrogen).

(2) Synthesis of Single Stranded DNA

1 μl of a mixed solution of all the first primers described in Table 2 and adjusted to predetermined concentration was added to an aqueous solution of 5 μg of the obtained total RNA, and incubated at 70° C. for 10 minutes. Next, after a mixed solution of 4 μl of 5×1st Strand buffer (a product of Invitrogen), 1 μl of 10 mM dNTP mixture, 2 μl of 100 mM DTT, 0.5 μl of RNase Inhibitor (a product of TOYOBO) and 2 μl of SuperScriptII (a product of Invitrogen) which is a reverse transcriptase were added, incubation was carried out at 42° C. for one hour.

TABLE 2

Primer Sequences used for Detection

| | |
|---|---|
| Chromosomal | First Primer |

| | | |
|---|---|---|
| Trans- | Gene | Sequence |

| | | | | |
|---|---|---|---|---|
| location | Name | Genbank | Primer | Listing |

| | | | | |
|---|---|---|---|---|
| t (9; 22) | BCR/ | X16416 | GGCCAGTGAATTGTAATACGACTCACTATAGGGAGGCGGGGATAATGGAGCGTGGTGAT | SEQ ID NO:52 |
| (q34; q11) | ABL_p210 | | | |
| | BCR/ | X16416 | GGCCAGTGAATTGTAATACGACTCACTATAGGGAGGCGGGGATAATGGAGCGTGGTGAT | SEQ ID NO:53 |
| | ABL_p190 | | | |
| | BCR/ | X16416 | GGCCAGTGAATTGTAATACGACTCACTATAGGGAGGCGGGGATAATGGAGCGTGGTGAT | SEQ ID NO:54 |
| | ABL_p230 | | | |
| t (15; 17) | PML/RARA | X06538 | GGCCAGTGAATTGTAATACGACTCACTATAGGGAGGCGGTGCACTTGGTGGAGAGTTCA | SEQ ID NO:55 |
| (q22; q21) | | | | |
| t (1; 19) | E2A/PBX1 | M86546 | GGCCAGTGAATTGTAATACGACTCACTATAGGGAGGCGGCCTCTTTGGCTTCCTCACTG | SEQ ID NO:56 |
| (q23; p13) | | | | |
| t (4; 11) | MLL/AF4 | L13773 | GGCCAGTGAATTGTAATACGACTCACTATAGGGAGGCGGCTGCTGCCCTTACTCTCTGG | SEQ ID NO:57 |
| (q21; q23) | | | | |
| t (9; 11) | MLL/AF9 | L13744 | GGCCAGTGAATTGTAATACGACTCACTATAGGGAGGCGGTGCATCCAGTTGTTATATCCTCA | SEQ ID NO:58 |
| (p22; q23) | | | | |
| t (8; 21) | AML1/ETO | D14289 | GGCCAGTGAATTGTAATACGACTCACTATAGGGAGGCGGTACTGGGCAGGGTTCTGTTT | SEQ ID NO:59 |
| (q22; q22) | | | | |
| t (12; 21) | TEL/AML1 | D43969 | GGCCAGTGAATTGTAATACGACTCACTATAGGGAGGCGGCCGATGTCTTCGAGGTTCTC | SEQ ID NO:60 |
| (p13; q22) | | | | |
| inv (16) | CBFB/ | D10667 | GGCCAGTGAATTGTAATACGACTCACTATAGGGAGGCGGGTAGCTGCTTGATGGCTTCC | SEQ ID NO:61 |
| (p13; q22) | MYH11 | | | |
| 1p32 | SIL/TAL1 | S53245 | GGCCAGTGAATTGTAATACGACTCACTATAGGGAGGCGGACAGGGTCCTTGCCAGTCTT | SEQ ID NO:62 |
| — | GAPD | X01677 | GGCCAGTGAATTGTAATACGACTCACTATAGGGAGGCGGGGTTGAGCACAGGGATACTTT | SEQ ID NO:63 |
| — | ACTB | X00351 | GGCCAGTGAATTGTAATACGACTCACTATAGGGAGGCGGGGTGTGCACTTTTATTCAACTGG | SEQ ID NO:64 |

| | |
|---|---|
| Chromosomal | Second Primer |

| | | |
|---|---|---|
| Trans- | Gene | Sequence |

| | | | | |
|---|---|---|---|---|
| location | Name | Genbank | Primer | Listing |

| | | | | |
|---|---|---|---|---|
| t (9; 22) | BCR/ | X02596 | TCTCTGCACCAAGCTCAAGA | SEQ ID NO:65 |
| (q34; q11) | ABL_p210 | | | |
| | BCR/ | X02596 | ACTACGAGGACGCCGAGTT | SEQ ID NO:66 |
| | ABL_p190 | | | |
| | BCR/ | X02596 | CTGAGCCAAACTGGAACGAG | SEQ ID NO:67 |
| | ABL_p230 | | | |
| t (15; 17) | PML/RARA | M73778 | GCCAGGTGGTAGCTCACG | SEQ ID NO:68 |
| (q22; q21) | | | | |
| t (1; 19) | E2A/PBX1 | M31222 | CATCTGCATCCTCCTTCTCC | SEQ ID NO:69 |
| (q23; p13) | | | | |
| t (4; 11) | MLL/AF4 | L04284 | TTGTGAAGAACGTGGTGGAC | SEQ ID NO:70 |
| (q21; q23) | | | | |
| t (9; 11) | MLL/AF9 | L04284 | TTGTGAAGAACGTGGTGGAC | SEQ ID NO:71 |
| (p22; q23) | | | | |
| t (8; 21) | AML1/ETO | D43969 | CCTTCGTACCCACAGTGCTT | SEQ ID NO:72 |
| (q22; q22) | | | | |
| t (12; 21) | TEL/AML1 | U11732 | ACACAGCCGGAGGTCATACT | SEQ ID NO:73 |
| (p13; q22) | | | | |
| inv (16) | CBFB/ | L20298 | GCAAGTTCGAGAACGAGGAG | SEQ ID NO:74 |
| (p13; q22) | MYH11 | | | |
| 1p32 | SIL/TAL1 | M74558 | GACCCCAACGTCCCAGAG | SEQ ID NO:75 |
| — | GAPD | X01677 | GATTCCACCCATGGCAAAT | SEQ ID NO:76 |
| — | ACTB | X00351 | GGCTACAGCTTCACCACCAC | SEQ ID NO:77 |

(3) Synthesis of Double Stranded DNA

150 μl of a mixed solution of 15 μl of 10×2nd Strand buffer, 15 μl of 906 mM KCl, 3 μl of 10 mM dNTP mixture, 4 μl of DNA polymerase I (a product of Invitrogen), 0.25 μl of Ribonuclease H (TaKaRa), 0.25 μl of DNA Ligase (TaKaRa), and 92.5 μl of DEPC water were added in a reaction tube and incubated at 16° C. for two hours, and 2 μl of T4 DNA polymerase (TOYOBO) was added and incubated for further 10 minutes. Then, the obtained double stranded DNA was purified by QIAquick PCR Purification Kit (a product of QIAGEN).

(4) Amplification of cRNA

After amplifying cRNA by using MEGAscript T7 in vitro RNA Transcription Kit (a product of Ambion) to a purified double stranded DNA solution, the obtained cRNA was purified using RNeasy Mini Kit (a product of QIAGEN).

(5) Synthesis of Single Stranded DNA using cRNA as a Template

1 μl of a mixed solution of all the second primers described in Table 2 and adjusted to predetermined concentration was added to the obtained cRNA aqueous solution, and incubated at 70° C. for 10 minutes. Next, after a mixed solution of 6 μl of 5×1st Strand buffer (a product of Invitrogen), 0.6 μl of dNTP mixture (25 mM for dATP, dGTP and dTTP, and 15 mM only for dCTP), 3 μl of 1 mM Cy5-dCTP, 3 μl of 100 mM DTT, 0.5 μl of RNase Inhibitor (a product of TOYOBO), 4.9 μl of DEPC water and 2 μl of SuperScriptII (a product of Invitrogen) which is a reverse transcriptase were added, incubation was carried out at 42° C. for one hour. Then, the obtained DNA was purified by QIAquick PCR Purification Kit (a product of QIAGEN).

4. Hybridization

To the purified DNA solution, appropriate amount of 20× Denhardt's solution (a product of SIGMA), 20×SSC and sodium dodecyl sulfate were added, and 24.5 μl of a hybridization solution was prepared so that the final concentration might be 2× Denhardt's solution, 4×SSC and 0.2% sodium dodecyl sulfate might be prepared. Then, after dropping the hybridization solution onto the glass on which the probes were immobilized and placing a cover glass was on it, hybridization reaction was carried out in an isothermic bath by allowing to leave the slide at 40° C. for 12 hours.

5. Detection of Signal Intensity

After immersing the slide into a mixed solution of a 10-fold diluted solution of 20×SSC and a 300-fold diluted solution of a 10% sodium dodecyl sulfate and removing the cover glass therefrom, the slide was washed with a 100-fold diluted solution of 20×SSC. Next, after removing the moisture on the slide using a centrifuge for microtiter plate, fluorescence intensity of each spot on which each of the probes was immobilized was measured using a scanner for micro arrays (Scan Array 5000; a product of PerkinElmer) and image analysis software (QuantArray, a product of PerkinElmer), and it was shown in Table 3. The type of the fusion gene determined from the pattern of the signal intensity of each of the obtained exon was also shown simultaneously.

### Example 2

In “(1) Preparation of RNA” of 3. Preparation of labeled products in the steps of Example 1, NB4 strain was used instead of K562 strain (a cell line derived from chronic myelocytic leukemia).

### Example 3

In “(1) Preparation of RNA” of 3. Preparation of labeled products in the steps of Example 1, RS4; 11 strain was used instead of K562 strain (a cell line derived from chronic myelocytic leukemia).

### Example 4

In “(1) Preparation of RNA” of 3. Preparation of labeled products in the steps of Example 1, THP-1 strain was used instead of K562 strain (a cell line derived from chronic myelocytic leukemia).

### Example 5

In “(1) Preparation of RNA” of 3. Preparation of labeled products in the steps of Example 1, Kasumi-1 strain was used instead of K562 strain (a cell line derived from chronic myelocytic leukemia).

### Example 6

In “(1) Preparation of RNA” of 3. Preparation of labeled products in the steps of Example 1, Reh strain was used instead of K562 strain (a cell line derived from chronic myelocytic leukemia).

### Example 7

In “(1) Preparation of RNA” of 3. Preparation of labeled products in the steps of Example 1, ME-1 strain was used instead of K562 strain (a cell line derived from chronic myelocytic leukemia).

### Example 8

In “(1) Preparation of RNA” of 3. Preparation of labeled products in the steps of Example 1, CEM strain was used instead of K562 strain (a cell line derived from chronic myelocytic leukemia).

### Example 9

In “(1) Preparation of RNA” of 3. Preparation of labeled products in the steps of Example 1, HL60 strain was used instead of K562 strain (a cell line derived from chronic myelocytic leukemia).

In the case of Examples 1 to 8, the fusion gene was detected applying the detection method of the fusion gene transcript of the present invention to the cell lines derived from leukemia in which the type of the expressed leukemia fusion gene was already known (Table 4). The types of the fusion gene detected from each of the cell lines are shown in Table 3, and the detected types are in agreement with the types already known, and the usefulness of the present invention was proved. On the other hand, it was known that the leukemia fusion gene is not expressed as for the cell line used in Example 9 and it was confirmed that the same result was obtained when the detection method of the present invention was used.

TABLE 3
Fluorescence Intensity after Hybridization

| | | Example 1; K562 | | | Example 2; NB4 | | | Example 3; RS4; 11 | | |
|---|---|---|---|---|---|---|---|---|---|---|
| | | Fluores- | | | Fluores- | | | Fluores- | | |
| Probe | | cence | Observed | Type of | cence | Observed | Type of | cence | Observed | Type of |
| Gene | | Inten- | PMT Gain | Fusion | Inten- | PMT Gain | Fusion | Inten- | PMT Gain | Fusion |
| Name | Exon | sity* | Value** | Gene | sity | Value | Gene | sity | Value | Gene |
| BCR/ABL | BCR_e13 | 20392 | 70 | BCR e14/ | — | — | — | — | — | — |
| p210 | BCR_e14 | 16002 | | ABL e2 | — | | | — | | |
| | ABL_e2 | 19485 | | (b3-a2) | — | | | — | | |
| | ABL_e3 | 21354 | | | — | | | — | | |
| BCR/ABL | BCR_e1 | 865 | 100 | BCR e1/ | — | — | — | — | — | — |
| p190 | ABL_e2 | 63550 | | ABL e2 | — | | | — | | |
| | ABL_e3 | 63550 | | (e1-a2) | — | | | — | | |
| BCR/ABL | BCR_e19 | — | — | — | — | — | — | — | — | — |
| p230 | BCR_e20 | — | | | — | | | — | | |
| | ABL_e2 | — | | | — | | | — | | |
| | ABL_e3 | — | | | — | | | — | | |
| PML/RARA | PML_e3 | — | — | — | 13585 | 80 | PML e6/ | — | — | — |
| | PML_e4 | — | | | 10076 | | RARA e3 | — | | |
| | PML_e5 | — | | | 14857 | | (bcr1) | — | | |
| | PML_e6 | — | | | 8258 | | | — | | |
| | PML_e6_1 | — | | | 13576 | | | — | | |
| | RARA_e3 | — | | | 15422 | | | — | | |
| MLL/AF4 | MLL_e8 | — | — | — | — | — | — | 56352 | 80 | MLL e10/ |
| | MLL_e9 | — | | | — | | | 40532 | | AF4 e4 |
| | MLL_e10 | — | | | — | | | 53252 | | |
| | MLL_e11 | — | | | — | | | — | | |
| | AF4_e4 | — | | | — | | | 10582 | | |
| | AF4_e5 | — | | | — | | | 51222 | | |
| | AF4_e6 | — | | | — | | | 6532 | | |
| | AF4_e7 | — | | | — | | | 47232 | | |
| MLL/AF9 | MLL_e8 | — | — | — | — | — | — | — | — | — |
| | MLL_e9 | — | | | — | | | — | | |
| | MLL_e10 | — | | | — | | | — | | |
| | MLL_e11 | — | | | — | | | — | | |
| | AF9_e5 | — | | | — | | | — | | |
| | AF9_e6 | — | | | — | | | — | | |
| | AF9_e7 | — | | | — | | | — | | |
| | AF9_e8 | — | | | — | | | — | | |
| | AF9_e9 | — | | | — | | | — | | |
| AML1/ETO | AML1_e5 | — | — | — | — | — | — | — | — | — |
| | ETO_e2 | — | | | — | | | — | | |
| TEL/AML1 | TEL_e5 | — | — | — | — | — | — | — | — | — |
| | AML1_e2 | — | | | — | | | — | | |
| | AML1_e3 | — | | | — | | | — | | |
| CBFB/MYH11 | CBFB_e4 | — | — | — | — | — | — | — | — | — |
| | CBFB_e5 | — | | | — | | | — | | |
| | MYH11_e7 | — | | | — | | | — | | |
| | MYH11_e7_1 | — | | | — | | | — | | |
| | MYH11_e8 | — | | | — | | | — | | |
| | MYH11_e9 | — | | | — | | | — | | |
| | MYH11_e10 | — | | | — | | | — | | |
| | MYH11_e10_ [symbol] | — | | | — | | | — | | |
| | MYH11_e11 | — | | | — | | | — | | |
| | MYH11_e12 | — | | | — | | | — | | |
| | MYH11_e13 | — | | | — | | | — | | |
| SIL/TAL1 | SIL_e1a | — | — | — | — | — | — | — | — | — |
| | SIL_e1b | — | | | — | | | — | | |
| | TAL1_e3 | — | | | — | | | — | | |
| | TAL1_e4 | — | | | — | | | — | | |
| E2A/PBX1 | E2A_e13 | — | — | — | — | — | — | — | — | — |
| | E2A_e13_1 | — | | | — | | | — | | |
| | PBX_e2 | — | | | — | | | — | | |
| GAPD | GAPD | 26351 | 50 | — | 22456 | 50 | — | 27584 | 50 | — |
| ACTB | ACTB | 48543 | 50 | — | 39777 | 50 | — | 36125 | 50 | — |

| | | Example 4; THP-1 | | | Example 5; Kasumi-1 | | | Example 6; Reh | | |
|---|---|---|---|---|---|---|---|---|---|---|
| | | Fluores- | | | Fluores- | | | Fluores- | | |
| Probe | | cence | Observed | | cence | Observed | Type of | cence | Observed | |
| Gene | | Inten- | PMT Gain | Type of | Inten- | PMT Gain | Fusion | Inten- | PMT Gain | Type of |
| Name | Exon | sity | Value | Fusion Gene | sity | Value | Gene | sity | Value | Fusion Gene |
| BCR/ABL | BCR_e13 | — | — | — | — | — | — | — | — | — |
| p210 | BCR_e14 | — | | | — | | | — | | |
| | ABL_e2 | — | | | — | | | — | | |
| | ABL_e3 | — | | | — | | | — | | |
| BCR/ABL | BCR_e1 | — | — | — | — | — | — | — | — | — |
| p190 | ABL_e2 | — | | | — | | | — | | |
| | ABL_e3 | — | | | — | | | — | | |
| BCR/ABL | BCR_e19 | — | — | — | — | — | — | — | — | — |
| p230 | BCR_e20 | — | | | — | | | — | | |
| | ABL_e2 | — | | | — | | | — | | |
| | ABL_e3 | — | | | — | | | — | | |
| PML/RARA | PML_e3 | — | — | — | — | — | — | — | — | — |
| | PML_e4 | — | | | — | | | — | | |
| | PML_e5 | — | | | — | | | — | | |
| | PML_e6 | — | | | — | | | — | | |
| | PML_e6_1 | — | | | — | | | — | | |
| | RARA_e3 | — | | | — | | | — | | |
| MLL/AF4 | MLL_e8 | — | — | — | — | — | — | — | — | — |
| | MLL_e9 | — | | | — | | | — | | |
| | MLL_e10 | — | | | — | | | — | | |
| | MLL_e11 | — | | | — | | | — | | |
| | AF4_e4 | — | | | — | | | — | | |
| | AF4_e5 | — | | | — | | | — | | |
| | AF4_e6 | — | | | — | | | — | | |
| | AF4_e7 | — | | | — | | | — | | |
| MLL/AF9 | MLL_e8 | 8444 | 80 | MLL e9/ | — | — | — | — | — | — |
| | MLL_e9 | 7655 | | AF9 e5 | — | | | — | | |
| | MLL_e10 | — | | | — | | | — | | |
| | MLL_e11 | — | | | — | | | — | | |
| | AF9_e5 | 7240 | | | — | | | — | | |
| | AF9_e6 | 7922 | | | — | | | — | | |
| | AF9_e7 | 6852 | | | — | | | — | | |
| | AF9_e8 | 4502 | | | — | | | — | | |
| | AF9_e9 | 7625 | | | — | | | — | | |
| AML1/ETO | AML1_e5 | — | — | — | 34282 | 60 | AML1 e5/ | — | — | — |
| | ETO_e2 | — | | | 31160 | | ETO e2 | — | | |
| TEL/AML1 | TEL_e5 | — | — | — | — | — | — | 27658 | 80 | TEL e5/ |
| | AML1_e2 | — | | | — | | | 9954 | | AML1 e2 |
| | AML1_e3 | — | | | — | | | 25864 | | |
| CBFB/ | CBFB_e4 | — | — | — | — | — | — | — | — | — |
| MYH11 | CBFB_e5 | — | | | — | | | — | | |
| | MYH11_e7 | — | | | — | | | — | | |
| | MYH11_e7_1 | — | | | — | | | — | | |
| | MYH11_e8 | — | | | — | | | — | | |
| | MYH11_e9 | — | | | — | | | — | | |
| | MYH11_e10 | — | | | — | | | — | | |
| | MYH11_e10_ [symbol] | — | | | — | | | — | | |
| | MYH11_e11 | — | | | — | | | — | | |
| | MYH11_e12 | — | | | — | | | — | | |
| | MYH11_e13 | — | | | — | | | — | | |
| SIL/TAL1 | SIL_e1a | — | — | — | — | — | — | — | — | — |
| | SIL_e1b | — | | | — | | | — | | |
| | TAL1_e3 | — | | | — | | | — | | |
| | TAL1_e4 | — | | | — | | | — | | |
| E2A/PBX1 | E2A_e13 | — | — | — | — | — | — | — | — | — |
| | E2A_e13_1 | — | | | — | | | — | | |
| | PBX_e2 | — | | | — | | | — | | |
| GAPD | GAPD | 20054 | 50 | — | 22548 | 50 | — | 29582 | 50 | — |
| ACTB | ACTB | 48582 | 50 | — | 38456 | 50 | — | 58762 | 50 | — |

| | | Example 7; ME-1 | | | Example 8; CEM | | | Example 9; HL60 | | |
|---|---|---|---|---|---|---|---|---|---|---|
| | | Fluores- | | | Fluores- | | | Fluores- | | |
| Probe | | cence | Observed | Type of | cence | Observed | Type of | cence | Observed | Type of |
| Gene | | Inten- | PMT Gain | Fusion | Inten- | PMT Gain | Fusion | Inten- | PMT Gain | Fusion |
| Name | Exon | sity | Value | Gene | sity | Value | Gene | sity | Value | Gene |
| BCR/ABL | BCR_e13 | — | — | — | — | — | — | — | — | — |
| p210 | BCR_e14 | — | | | — | | | — | | |
| | ABL_e2 | — | | | — | | | — | | |
| | ABL_e3 | — | | | — | | | — | | |
| BCR/ABL | BCR_e1 | — | — | — | — | — | — | — | — | — |
| p190 | ABL_e2 | — | | | — | | | — | | |
| | ABL_e3 | — | | | — | | | — | | |
| BCR/ABL | BCR_e19 | — | — | — | — | — | — | — | — | — |
| p230 | BCR_e20 | — | | | — | | | — | | |
| | ABL_e2 | — | | | — | | | | | |
| | ABL_e3 | — | | | — | | | — | | |
| PML/RARA | PML_e3 | — | — | — | — | — | — | — | — | — |
| | PML_e4 | — | | | — | | | — | | |
| | PML_e5 | — | | | — | | | — | | |
| | PML_e6 | — | | | — | | | — | | |
| | PML_e6_1 | — | | | — | | | — | | |
| | RARA_e3 | — | | | — | | | — | | |
| MLL/AF4 | MLL_e8 | — | — | — | — | — | — | — | — | — |
| | MLL_e9 | — | | | — | | | — | | |
| | MLL_e10 | — | | | — | | | — | | |
| | MLL_e11 | — | | | — | | | — | | |
| | AF4_e4 | — | | | — | | | — | | |
| | AF4_e5 | — | | | — | | | — | | |
| | AF4_e6 | — | | | — | | | — | | |
| | AF4_e7 | — | | | — | | | — | | |
| MLL/AF9 | MLL_e8 | — | — | — | — | — | — | — | — | — |
| | MLL_e9 | — | | | — | | | — | | |
| | MLL_e10 | — | | | — | | | — | | |
| | MLL_e11 | — | | | — | | | — | | |
| | AF9_e5 | — | | | — | | | — | | |
| | AF9_e6 | — | | | — | | | — | | |
| | AF9_e7 | — | | | — | | | — | | |
| | AF9_e8 | — | | | — | | | — | | |
| | AF9_e9 | — | | | — | | | — | | |
| AML1/ETO | AML1_e5 | — | — | — | — | — | — | — | — | — |
| | ETO_e2 | — | | | — | | | — | | |
| TEL/AML1 | TEL_e5 | — | — | — | — | — | — | — | — | — |
| | AML1_e2 | — | | | — | | | — | | |
| | AML1_e3 | — | | | — | | | — | | |
| CBFB/ | CBFB_e4 | 10598 | 70 | CBFB e5/ | — | — | — | — | — | — |
| MYH11 | CBFB_e5 | 12464 | | MYH11 e12 | — | | | — | | |
| | MYH11_e7 | — | | | — | | | — | | |
| | MYH11_e7_1 | — | | | — | | | — | | |
| | MYH11_e8 | — | | | — | | | — | | |
| | MYH11_e9 | — | | | — | | | — | | |
| | MYH11_e10 | — | | | — | | | — | | |
| | MYH11_e10_1 | — | | | — | | | — | | |
| | MYH11_e11 | — | | | — | | | — | | |
| | MYH11_e12 | 9978 | | | — | | | — | | |
| | MYH11_e13 | 11567 | | | — | | | — | | |
| SIL/TAL1 | SIL_e1a | — | — | — | 27653 | 80 | SIL e1a | — | — | — |
| | SIL_e1b | — | | | 15342 | | (and SIL | — | | |
| | TAL1_e3 | — | | | 28654 | | e1b)/ | — | | |
| | TAL1_e4 | — | | | 26532 | | TAL e3 | — | | |
| E2A/PBX1 | E2A_e13 | — | — | | — | | — | — | | |
| | E2A_e13_1 | — | | — | — | — | | — | — | — |
| | PBX_e2 | — | | | — | | | — | | |
| GAPD | GAPD | 23585 | 50 | — | 28857 | 50 | — | 19578 | 50 | — |
| ACTB | ACTB | 45789 | 50 | — | 40528 | 50 | — | 35284 | 50 | — |

*Fluorescence Intensity: Expressed as “_” when the intensity is no more than 300.
**Observed PMT Gain Value: Photo multiplier set value in scanner when scanning.

TABLE 4
Type of Detected Fusion Gene

| | | | Number |
|---|---|---|---|
| Chromosome | Name of | Type of Fusion | of |
| Translocation | Fusion Gene | Gene | Sumple |
| t(9; 22) | BCR/ABL_p210 | BCR e14/ABL e2(b3-a2) | 1 |
| (q34; q11) | BCR/ABL_p190 | BCR e1/ABL e2(e1-a2) | 2 |
| | BCR/ABL_p230 | — | — |
| t(15; 17) | PML/RARA | PML e6/RARA e3(bcr1) | 2 |
| (q22; q21) | | PML e3/RARA e3(bcr3) | 1 |
| t(1; 19) | E2A/PBX1 | — | — |
| (q23; p13) | | | |
| t(4; 11) | MLL/AF4 | MLL e10/AF4 e4 | 1 |
| (q21; q23) | | | |
| t(9; 11) | MLL/AF9 | MLL e9/AF9 e5 | 1 |
| (p22; q23) | | | |
| t(8; 21) | AML1/ETO | — | — |
| (q22; q22) | | | |
| t(12; 21) | TEL/AML1 | — | — |
| (p13; q22) | | | |
| inv(16) | CBFB/MYH11 | — | — |
| (p13; q22) | | | |
| 1p32 | SIL/TAL1 | — | — |

### Example 10

In “(1) Preparation of RNA” of 3. Preparation of labeled products in the steps of Example 1, total RNA is extracted from white blood cells in the peripheral blood extracted from a patient of hematopoietic tumor with informed consent using QIAamp Blood Mini Kit of QIAGEN.

Detection of a fusion gene is conducted on the extracted total RNA using the detection method of the fusion gene transcripts of the present invention. For the sample in which the leukemia fusion gene is detected, confirmation experiments are conducted by the Real-time PCR method (see JP Patent Publication (Kokai) No. 2002-136300, etc.) or NASBA method.

### INDUSTRIAL APPLICABILITY

The method for detecting fusion gene transcripts of the present invention can detect two or more fusion genes with a high throughput at a time. Therefore, it can be widely used from basic research to clinical application such as researches, diagnosis, selection of a therapeutic method, etc. of diseases closely associated with generation of fusion genes such as leukemia.

[Free Text in Sequence Listing]

SEQ ID NOs: 1-51—Description of artificial sequence: synthetic DNA (probe)

SEQ ID NOs: 52-64—Description of artificial sequence: synthetic DNA (primer)

SEQ ID NOs: 65-77—Description of artificial sequence: synthetic DNA (primer)

SEQ ID NO: 78—Description of artificial sequence: sequence derived from T7RNA promoter

SEQ ID NO: 79—Description of artificial sequence: sequence derived from T3RNA promoter

SEQ ID NO: 80—Description of artificial sequence: sequence derived from SP6RNA promoter

What is claimed is:

1. A method for detecting fusion gene transcripts wherein the method comprises allowing at least two or more of probes, each of which contains a partial base sequence of exons which sandwich the breakpoint of a fusion gene or complementary base sequence thereof, and each of which is immobilized on a support, to hybridize with a sample containing a nucleic acid derived from a fusion gene, thereby allowing the detection of two or more fusion genes at a time.

2. The method according to claim 1 wherein two or more fusion genes are identified which are identical in the combination of translocated genes but different from each other in the translocated positions in the genes by analyzing the signal intensity from the nucleic acid hybridized with each probe.

3. The method according to claim 1 wherein the method comprises, as the step of preparing a sample containing the nucleic acids derived from said fusion gene, the following steps (1) to (4):
(1) a step of synthesizing a single stranded DNA by subjecting RNA obtained from a specimen from subject to reverse transcription reaction;
(2) a step of synthesizing a double stranded DNA by using said single stranded DNA as a template;
(3) a step of amplifying cRNA using RNA polymerase by using said double stranded DNA as a template; and
(4) a step of synthesizing a single stranded DNA by performing reverse transcription reaction by using said cRNA as a template.

4. The method according to claim 3 wherein a primer containing a base sequence complementary to a sequence of at least ten or more contiguous bases which exist on the 3′ side from the breakpoint of the fusion gene is used in the reverse transcription reaction of said step (1).

5. The method according to claim 4 wherein said primer further comprises a promoter sequence of RNA polymerase.

6. The method according to claim 3 wherein a primer containing a base sequence complementary to a sequence of at least ten or more contiguous bases which exist on the 5′ side from the breakpoint of the fusion gene is used in the reverse transcription reaction of said step (4).

7. The method according to claim 3 wherein the primer of said step (1) comprises a base sequence as shown in any one of SEQ ID NOs: 52 to 64 and the primer of said step (4) comprises a base sequence as shown in any one of SEQ ID NOs: 65 to 77.

8. The method according to claim 1 wherein said probe comprises a base sequence as shown in any one of SEQ ID NOs: 1 to 51.

9. An in vitro diagnostic method of leukemia using a method according to claim 1.

10. A kit for detecting fusion gene transcripts comprising the following (a) to (c):
(a) a support on which two or more probes, each of which contains a partial base sequence of the exons which sandwich the breakpoint of a fusion gene or complementary base sequences thereof are immobilized;
(b) a first primer containing a base sequence complementary to a base sequence of at least ten or more contiguous bases which exist on the 3′ side from the breakpoint of the fusion gene; and
(c) a second primer containing a base sequence of at least ten or more contiguous bases which exist on the 5′ side from the breakpoint of the fusion gene.

11. The kit according to claim 10 wherein said probe comprises a base sequence as shown in any one of SEQ ID NOs: 1 to 51, said first primer comprises a base sequence as shown in any one of SEQ ID NOs: 52 to 64 and said second primer comprises a base sequence as shown in any one of SEQ ID NOs: 65 to 77.


SEQUENCE LISTING (80 sequences)

SEQ ID NO: 1 - DNA, 80 bp, Artificial Sequence
  Description of Artificial SequenceSynthetic DNA (probe)
cttccttattgatggtcagcggaatgctgtggacagtctggagtttcacacacgagttggtcagcatctgcagctccacg

SEQ ID NO: 2 - DNA, 80 bp, Artificial Sequence
  Description of Artificial SequenceSynthetic DNA (probe)
ttgaactctgcttaaatccagtggctgagtggacgatgacattcagaaacccatagagccccggagactcatcatttttt

SEQ ID NO: 3 - DNA, 80 bp, Artificial Sequence
  Description of Artificial SequenceSynthetic DNA (probe)
tccactggccacaaaatcatacagtgcaacgaaaaggttggggtcattttcactgggtccagcgagaaggttttccttgg

SEQ ID NO: 4 - DNA, 80 bp, Artificial Sequence
  Description of Artificial SequenceSynthetic DNA (probe)
actggcgtgatgtagttgcttgggacccagccttggccatttttggtttgggcttcacaccattccccattgtgattata

SEQ ID NO: 5 - DNA, 80 bp, Artificial Sequence
  Description of Artificial SequenceSynthetic DNA (probe)
tgctgctgtcgaaggactgttgcgagttctgcgagggagagcggcttttgtcccggaacatgcggtaggtggtggggctt

SEQ ID NO: 6 - DNA, 80 bp, Artificial Sequence
  Description of Artificial SequenceSynthetic DNA (probe)
tgacgtcgaaggctgccttcagtgcctggatgtccgtggccacaccggacacgcggtagatgcccacctcctccatgcct

SEQ ID NO: 7 - DNA, 80 bp, Artificial Sequence
  Description of Artificial SequenceSynthetic DNA (probe)
gctcacggaagtacagcttcagcgtgcctgcgatggcgttcacgtccatctcgctcatcatcaccgacacatccttgtta

SEQ ID NO: 8 - DNA, 80 bp, Artificial Sequence
  Description of Artificial SequenceSynthetic DNA (probe)
ctttcccctgggtgatgcaagagctgaggtcctgcaggcgcaccttgaactcgtcgaagccatcggtgcgcacggcagct

SEQ ID NO: 9 - DNA, 80 bp, Artificial Sequence
  Description of Artificial SequenceSynthetic DNA (probe)
gcaggtcaacgtcaatagggtccctgggagtgctggcagcctctgggctggctttcttggatacagctgcattttttttt

SEQ ID NO: 10 - DNA, 80 bp, Artificial Sequence
  Description of Artificial SequenceSynthetic DNA (probe)
gcctttgatggagaaggcgtacactggcacggggtgtgccccgggcactgactgtaccacagccataggctgggcttcag

SEQ ID NO: 11 - DNA, 80 bp, Artificial Sequence
  Description of Artificial SequenceSynthetic DNA (probe)
tgaccttcctggggcactgggtctggctgcacttcctcttctgggctgtcgttgtattggagacatcttttttttttttt

SEQ ID NO: 12 - DNA, 80 bp, Artificial Sequence
  Description of Artificial SequenceSynthetic DNA (probe)
gttgctgttgggcaggaagacctcacttcctatgacggggctcctggggctaggcggtccatccaggtggggtggtgaga

SEQ ID NO: 13 - DNA, 80 bp, Artificial Sequence
  Description of Artificial SequenceSynthetic DNA (probe)
gcttgtagatgcggggtagagggggtggcgagggagggctgggcactatctcttcagaactgctgctctgggtctcaatg

SEQ ID NO: 14 - DNA, 80 bp, Artificial Sequence
  Description of Artificial SequenceSynthetic DNA (probe)
actgtaggagtcgggaggccgagacaggtcagggagggtgcctggctggctggggagggccgcgtggttgtgcatgaggc

SEQ ID NO: 15 - DNA, 80 bp, Artificial Sequence
  Description of Artificial SequenceSynthetic DNA (probe)
aggttccaggtgaccgaacactttcatttttttttttttttttttttttttttttttttttttttttttttttttttttt

SEQ ID NO: 16 - DNA, 80 bp, Artificial Sequence
  Description of Artificial SequenceSynthetic DNA (probe)
acgtgggtggtgaactcgttgcaggcctgctcgtatttctccagctccgtatggtagatttgtctgatctgtgagagttt

SEQ ID NO: 17 - DNA, 80 bp, Artificial Sequence
  Description of Artificial SequenceSynthetic DNA (probe)
ctgattctggtggtggaggctgctttttcttgggctcactaggagtggttttgggaacttcttttcttggcggtcctgta

SEQ ID NO: 18 - DNA, 80 bp, Artificial Sequence
  Description of Artificial SequenceSynthetic DNA (probe)
cttttcttttggtttttgttttacagggatacttgggcggggagccacttttttctgtttgctctgctctggactttttt

SEQ ID NO: 19 - DNA, 80 bp, Artificial Sequence
  Description of Artificial SequenceSynthetic DNA (probe)
tgcttagaactattgccattggagagagtgctgaggatgttcaaagtgcctgcattctcctgcttattgaccggaggtgg

SEQ ID NO: 20 - DNA, 80 bp, Artificial Sequence
  Description of Artificial SequenceSynthetic DNA (probe)
tgcccactactggcacagagaaagcaaaccaccctgggtgttataggaacagaagtcaagattcctaagcctcccatctc

SEQ ID NO: 21 - DNA, 80 bp, Artificial Sequence
  Description of Artificial SequenceSynthetic DNA (probe)
ccttcagaatctcttcaacacaatggacttcattggagtaggtctgtttttttttttttttttttttttttttttttttt

SEQ ID NO: 22 - DNA, 80 bp, Artificial Sequence
  Description of Artificial SequenceSynthetic DNA (probe)
ttgtagggaaaggaaacttggatggctcagctgtactaggcgtatgtattgctgtcaaaggaggcggccatgaatgggtc

SEQ ID NO: 23 - DNA, 80 bp, Artificial Sequence
  Description of Artificial SequenceSynthetic DNA (probe)
ttttggttttgggttacagaactgacatgctgagagtttttttttttttttttttttttttttttttttttttttttttt

SEQ ID NO: 24 - DNA, 80 bp, Artificial Sequence
  Description of Artificial SequenceSynthetic DNA (probe)
tcactgtcactgtcctcactgtcactgagctgaaggtcgtcttcgagcatggatgacgttccttgctgagaatttgagtg

SEQ ID NO: 25 - DNA, 80 bp, Artificial Sequence
  Description of Artificial SequenceSynthetic DNA (probe)
gaccttgttgcctggtctgggatggtgtgaagctggagctggagctggagctggagctggcaggactgggttgttcagat

SEQ ID NO: 26 - DNA, 80 bp, Artificial Sequence
  Description of Artificial SequenceSynthetic DNA (probe)
actctgcgacttcggctgcctcctctatttacaggcctctccatttcagagtcattgtcgttatcctccacttcatctga

SEQ ID NO: 27 - DNA, 80 bp, Artificial Sequence
  Description of Artificial SequenceSynthetic DNA (probe)
gttggtttttagtaagggtggtggaggttcgtgatgtaggggtgaagaagcagaactgctttcactatcgctgccatcac

SEQ ID NO: 28 - DNA, 80 bp, Artificial Sequence
  Description of Artificial SequenceSynthetic DNA (probe)
ccttgtcacattcaccattctttatttgcttatctgatttgctttgctttattggacttttcacttcaagaatttttttt

SEQ ID NO: 29 - DNA, 80 bp, Artificial Sequence
  Description of Artificial SequenceSynthetic DNA (probe)
aaggttcacgatctgctgcagaatgtgtctttctctcaatgtcattaaccttctgtgaagctctaccagttcatctaggt

SEQ ID NO: 30 - DNA, 80 bp, Artificial Sequence
  Description of Artificial SequenceSynthetic DNA (probe)
cactgtgattttgatggctctgtggtaggtggcgacttgcggtgggtttgtgaagacagtgatggtcagagtgaagcttt

SEQ ID NO: 31 - DNA, 80 bp, Artificial Sequence
  Description of Artificial SequenceSynthetic DNA (probe)
ttgtcggtgtaaatgaactggttcttggagctccttgagtagttgggggaggtggcattgttggaggagtcagcctagat

SEQ ID NO: 32 - DNA, 80 bp, Artificial Sequence
  Description of Artificial SequenceSynthetic DNA (probe)
gtgatttgtcgtgataggtgacctggagcggtgcaacagttcaatggtgggagggttatggtgcacattatccacggatg

SEQ ID NO: 33 - DNA, 80 bp, Artificial Sequence
  Description of Artificial SequenceSynthetic DNA (probe)
tcgtggacgtctctagaaggattcattccaagtatgcatttttttttttttttttttttttttttttttttttttttttt

SEQ ID NO: 34 - DNA, 80 bp, Artificial Sequence
  Description of Artificial SequenceSynthetic DNA (probe)
tcggtgcgcaccagctcgcccgggtggtcggccagcacctccaccatgctgcggtcgccgctcctcagcttgccggccag

SEQ ID NO: 35 - DNA, 80 bp, Artificial Sequence
  Description of Artificial SequenceSynthetic DNA (probe)
tgggctcgctcctcatcaaactccagacagcccataccatccagtctttggagatcaatccagcctttccagataacaca

SEQ ID NO: 36 - DNA, 80 bp, Artificial Sequence
  Description of Artificial SequenceSynthetic DNA (probe)
ctccatttcctcccgatgagacctgtctctatcttcaaattcgcgtgtccttctccgagcctcttcaaaggcctgttgtg

SEQ ID NO: 37 - DNA, 80 bp, Artificial Sequence
  Description of Artificial SequenceSynthetic DNA (probe)
tggcccaggacccgcagctccccggccaggtctgcgttctctttctccagcgtctgcttattcttgtctaggttcgcctt

SEQ ID NO: 38 - DNA, 80 bp, Artificial Sequence
  Description of Artificial SequenceSynthetic DNA (probe)
tctgcagcttgtggactttgtcattgagctccgcccgggcccgctccccatcgctgcacttggactgcagctcctgcacc

SEQ ID NO: 39 - DNA, 80 bp, Artificial Sequence
  Description of Artificial SequenceSynthetic DNA (probe)
actgagggacgccacgtccttggccagcttaatggccttcccctcggcctcgttaagcatccctgtgacgctctcaactt

SEQ ID NO: 40 - DNA, 80 bp, Artificial Sequence
  Description of Artificial SequenceSynthetic DNA (probe)
gtcttgcaggctgttccgctcctcctccagctggcgcagcttcgtagacacgttgagcttctgccgggtttcttcttgaa

SEQ ID NO: 41 - DNA, 80 bp, Artificial Sequence
  Description of Artificial SequenceSynthetic DNA (probe)
tcttcttcccctcttccagagcttccacggtgctggcaaagtcctgcagcttcttcttcgagtcggagattttttttttt

SEQ ID NO: 42 - DNA, 80 bp, Artificial Sequence
  Description of Artificial SequenceSynthetic DNA (probe)
ggttgtccaaatcaacaaccaggtcgtccagctcctgctgaagcctgttcttggtcttttccagtttatcataagcggcc

SEQ ID NO: 43 - DNA, 80 bp, Artificial Sequence
  Description of Artificial SequenceSynthetic DNA (probe)
gtttccttctccctggcttctgcctcagctctgtccctctcatccgcgtatttggaagagatgtttttctcctcggctaa

SEQ ID NO: 44 - DNA, 80 bp, Artificial Sequence
  Description of Artificial SequenceSynthetic DNA (probe)
ctcctcttctcctcattctgctcgtcccgggcttggagatccctttcgaactggcccttgagcgcctgcatgttgacttc

SEQ ID NO: 45 - DNA, 80 bp, Artificial Sequence
  Description of Artificial SequenceSynthetic DNA (probe)
ttcaggtccccttccagcttcttctttgctgcagctgccagggcacgttcgtttcgctcgtcttccagttccgtctcata

SEQ ID NO: 46 - DNA, 80 bp, Artificial Sequence
  Description of Artificial SequenceSynthetic DNA (probe)
gagccggctccaaggagcgccaccgccgactccggccccgcctctgggacgttggggtcgcgggaactacgtgtttaagt

SEQ ID NO: 47 - DNA, 80 bp, Artificial Sequence
  Description of Artificial SequenceSynthetic DNA (probe)
cagcattttgggaggccgaggcgggtggatcacctgaagtcaggatttccagaccagcccgaccaacatggtgaaacccc

SEQ ID NO: 48 - DNA, 80 bp, Artificial Sequence
  Description of Artificial SequenceSynthetic DNA (probe)
catatttagagagaccggcccctctgaataggatctccactccgccggaaaggggcggaagccgaggaagaggatgcaca

SEQ ID NO: 49 - DNA, 80 bp, Artificial Sequence
  Description of Artificial SequenceSynthetic DNA (probe)
cgcgtcgcggccctttaagtctctcgcggcgccgcccccaccggcagggccgccccccgggcctccgcgcgcgcccagtt

SEQ ID NO: 50 - DNA, 80 bp, Artificial Sequence
  Description of Artificial SequenceSynthetic DNA (probe)
atactttattagatggatacatgacaaggtgcggctcccataggcccctcccctcttcaaggggtctacatggcaactgt

SEQ ID NO: 51 - DNA, 80 bp, Artificial Sequence
  Description of Artificial SequenceSynthetic DNA (probe)
tgcattacataatttacacgaaagcaatgctatcacctcccctgtgtggacttgggagaggactgggccattctccttag

SEQ ID NO: 52 - DNA, 59 bp, Artificial Sequence
  Description of Artificial SequenceSynthetic DNA (primer)
ggccagtgaattgtaatacgactcactatagggaggcggggataatggagcgtggtgat

SEQ ID NO: 53 - DNA, 59 bp, Artificial Sequence; identical to SEQ ID NO: 52
  Description of Artificial SequenceSynthetic DNA (primer)

SEQ ID NO: 54 - DNA, 59 bp, Artificial Sequence; identical to SEQ ID NO: 52
  Description of Artificial SequenceSynthetic DNA (primer)

SEQ ID NO: 55 - DNA, 59 bp, Artificial Sequence
  Description of Artificial SequenceSynthetic DNA (primer)
ggccagtgaattgtaatacgactcactatagggaggcggtgcacttggtggagagttca

SEQ ID NO: 56 - DNA, 59 bp, Artificial Sequence
  Description of Artificial SequenceSynthetic DNA (primer)
ggccagtgaattgtaatacgactcactatagggaggcggcctctttggcttcctcactg

SEQ ID NO: 57 - DNA, 59 bp, Artificial Sequence
  Description of Artificial SequenceSynthetic DNA (primer)
ggccagtgaattgtaatacgactcactatagggaggcggctgctgcccttactctctgg

SEQ ID NO: 58 - DNA, 62 bp, Artificial Sequence
  Description of Artificial SequenceSynthetic DNA (primer)
ggccagtgaattgtaatacgactcactatagggaggcggtgcatccagttgttatatcctca

SEQ ID NO: 59 - DNA, 59 bp, Artificial Sequence
  Description of Artificial SequenceSynthetic DNA (primer)
ggccagtgaattgtaatacgactcactatagggaggcggtactgggcagggttctgttt

SEQ ID NO: 60 - DNA, 59 bp, Artificial Sequence
  Description of Artificial SequenceSynthetic DNA (primer)
ggccagtgaattgtaatacgactcactatagggaggcggccgatgtcttcgaggttctc

SEQ ID NO: 61 - DNA, 59 bp, Artificial Sequence
  Description of Artificial SequenceSynthetic DNA (primer)
ggccagtgaattgtaatacgactcactatagggaggcgggtagctgcttgatggcttcc

SEQ ID NO: 62 - DNA, 59 bp, Artificial Sequence
  Description of Artificial SequenceSynthetic DNA (primer)
ggccagtgaattgtaatacgactcactatagggaggcggacagggtccttgccagtctt

SEQ ID NO: 63 - DNA, 60 bp, Artificial Sequence
  Description of Artificial SequenceSynthetic DNA (primer)
ggccagtgaattgtaatacgactcactatagggaggcggggttgagcacagggatacttt

SEQ ID NO: 64 - DNA, 62 bp, Artificial Sequence
  Description of Artificial SequenceSynthetic DNA (primer)
ggccagtgaattgtaatacgactcactatagggaggcggggtgtgcacttttattcaactgg

SEQ ID NO: 65 - DNA, 20 bp, Artificial Sequence
  Description of Artificial SequenceSynthetic DNA (primer)
tctctgcaccaagctcaaga

SEQ ID NO: 66 - DNA, 19 bp, Artificial Sequence
  Description of Artificial SequenceSynthetic DNA (primer)
actacgaggacgccgagtt

SEQ ID NO: 67 - DNA, 20 bp, Artificial Sequence
  Description of Artificial SequenceSynthetic DNA (primer)
ctgagccaaactggaacgag

SEQ ID NO: 68 - DNA, 18 bp, Artificial Sequence
  Description of Artificial SequenceSynthetic DNA (primer)
gccaggtggtagctcacg

SEQ ID NO: 69 - DNA, 20 bp, Artificial Sequence
  Description of Artificial SequenceSynthetic DNA (primer)
catctgcatcctccttctcc

SEQ ID NO: 70 - DNA, 20 bp, Artificial Sequence
  Description of Artificial SequenceSynthetic DNA (primer)
ttgtgaagaacgtggtggac

SEQ ID NO: 71 - DNA, 20 bp, Artificial Sequence
  Description of Artificial SequenceSynthetic DNA (primer)
ttgtgaagaacgtggtggac

SEQ ID NO: 72 - DNA, 20 bp, Artificial Sequence
  Description of Artificial SequenceSynthetic DNA (primer)
ccttcgtacccacagtgctt

SEQ ID NO: 73 - DNA, 20 bp, Artificial Sequence
  Description of Artificial SequenceSynthetic DNA (primer)
acacagccggaggtcatact

SEQ ID NO: 74 - DNA, 20 bp, Artificial Sequence
  Description of Artificial SequenceSynthetic DNA (primer)
gcaagttcgagaacgaggag

SEQ ID NO: 75 - DNA, 18 bp, Artificial Sequence
  Description of Artificial SequenceSynthetic DNA (primer)
gaccccaacgtcccagag

SEQ ID NO: 76 - DNA, 19 bp, Artificial Sequence
  Description of Artificial SequenceSynthetic DNA (primer)
gattccacccatggcaaat

SEQ ID NO: 77 - DNA, 20 bp, Artificial Sequence
  Description of Artificial SequenceSynthetic DNA (primer)
ggctacagcttcaccaccac

SEQ ID NO: 78 - DNA, 25 bp, Artificial Sequence
  Description of Artificial Sequence sequence derived from T7RNA promoter
aattgtaatacgactcactataggg

SEQ ID NO: 79 - DNA, 17 bp, Artificial Sequence
  Description of Artificial Sequence sequence derived from T3RNA promoter
attaaccctcactaaag

SEQ ID NO: 80 - DNA, 17 bp, Artificial Sequence
  Description of Artificial Sequence sequence derived from SP6RNA promoter
atttaggtgacactata